(19) United States
(12) Patent Application Publication
(10) Pub. No.: US 2004/0184952 A1
(43) Pub. Date: Sep. 23, 2004

(54) Method for sterilizing / disinfecting the water circuit of a dental unit and a dental unit implementing the method

(75) Inventor: Franco Castellini, Bologna (IT)

Correspondence Address: Richard J. Minnich, Esq., Fay, Sharpe, Fagan, Minnich & McKee, LLP, Seventh Floor, 1100 Superior Avenue, Cleveland, OH, 44114-2518, US

(73) Assignee: CASTELLINI S.p.A.

(21) Appl. No.: 10/394,878

(22) Filed: Mar. 21, 2003

(51) Int. Cl. A01N 1/00; C23F 11/00

(52) U.S. Cl. 422/28; 422/1; 422/3

(57) ABSTRACT

A method for sterilizing/disinfecting the water circuit (1) of a dental unit (100) at least comprising medical instruments, such as dental handpieces (2), and a water circuit (1) for supplying a fluid from a main supply (3) through corresponding circuit legs or branches (1a, 1b, 1c, 1s); the method comprising at least the following steps: introducing the disinfectant/sterilizing liquid in the water circuit (1) for a predetermined length of time and draining the disinfectant/sterilizing liquid out of the circuit (1) through the circuit legs or branches (1a, 1b, 1c, 1s). The step of introducing the disinfectant/sterilizing liquid is accompanied by a step of heating the liquid to a predetermined temperature higher than ambient temperature, using suitable means (4).

### BACKGROUND OF THE INVENTION

The present invention relates to a method for sterilizing/disinfecting the water circuit of a dental unit and to a dental unit implementing the method.

In the manufacturing of dental units, one of the fundamental parts, if not the “heart”, of a dental unit is its water and air circuit, where the water line supplies fluids used by dental equipment and patients (water or physiological saline for tumblers and handpieces), or consumer units (swilling water for the spittoon), while the air line is used for certain items of equipment (air spray handpieces, cooling air and driving air).

With increases in general standards of hygiene and with dental apparatus and equipment becoming more and more “fragile”, several design solutions have been found for the water and air circuits of dental units, not only to guarantee their efficient operation and durability but also to maintain the sterility of the conduits during patient treatments.

Considering that the basic structure of these fluid circuits comprises a first main line supplying water from the mains, and a second main line supplying air from an external source (compressor), each of which has a plurality of branches leading to the operating and accessory equipment of the dental unit, different systems have been designed on the basis of different methods aimed at improving the functioning and disinfection of these fluid lines or parts of them.

In particular, the present specification focuses attention on the water line which is disinfected according to two different methods, one with a continuous cycle and the other with a discontinuous cycle, and both requiring additional devices to be fitted to the basic structure of the circuit.

The present specification is concerned in particular with the solutions based on the discontinuous disinfection/sterilization cycle. In this type of cycle, as disclosed in patent publications EP-111.249 and EP-317.521 (the latter being by the present Applicant), the mains water supply is shut off, and a “dedicated” branch equipped with an independent tank is used to feed sterilizing liquid into the conduits that supply water to the hand pieces.

After a preset time, depending on the quality of disinfection/sterilization required and the properties of the sterilizing liquid, the line is opened again and the sterilizing liquid drained out.

The drainage of the sterilizing liquid is performed by flushing water (or a user fluid) supplied by the main line (or by a dedicated line) and opening the control valves on the handpieces so that the water or user fluid expels the sterilizing liquid, rinses the water line and flows out into an appropriate drain.

Although this method, which has been used on dental units for some time, has proved to be very effective and practical, the Applicant, has found, after in-depth research conducted in accordance with its policy of continual improvement of dental unit sanitizing procedures, that the sanitizing product or products used require a considerable amount of time to take action.

The aim of the present invention is to overcome the above mentioned drawback by providing a sterilizing/disinfecting method, and a dental unit that implements the method, that is practical and fast and, at the same, gives excellent results in sanitizing the water circuit of the dental unit.

### SUMMARY OF THE INVENTION

The above mentioned aim is achieved in a method for sterilizing/disinfecting the water circuit of a dental unit comprising medical instruments, such as dental handpieces, and a water circuit for supplying fluid from a main supply, the method comprising at least the following steps: introducing the disinfectant/sterilizing liquid in the water circuit for a predetermined length of time and draining the disinfectant/sterilizing liquid out of the circuit through the circuit legs or branches; the step of introducing the disinfectant/sterilizing liquid being accompanied by a step of heating the liquid to a predetermined temperature higher than ambient temperature, using suitable means.

### BRIEF DESCRIPTION OF THE DRAWINGS

The technical characteristics of the invention, with reference to the above aims, are clearly described in the claims below and its advantages are apparent from the detailed description which follows, with reference to the accompanying drawings which illustrate a preferred embodiment of the invention provided merely by way of example without restricting the scope of the inventive concept, and in which:

FIG. 1 is a diagram of the water circuit of a dental unit implementing the sterilizing/disinfecting method according to the present invention;

FIG. 2 is a schematic detail view, with some parts cut away in order to better illustrate others, of the dental unit implementing the sterilizing/disinfecting method according to the invention.

### DESCRIPTION OF THE PREFERRED EMBODIMENTS

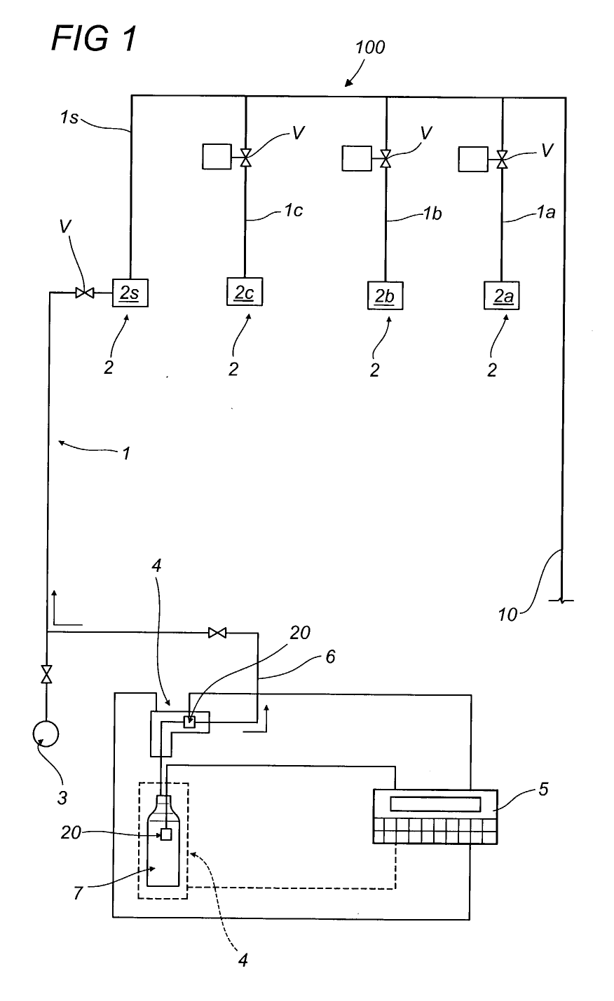

With reference to the accompanying drawings, in particular FIG. 1, the method according to the invention is used to sterilize/disinfect the water circuit 1 of a dental unit that is labeled 100 in its entirety.

The dental unit 100 comprises, at least insofar as is relevant to the present invention, medical instruments, such as dental handpieces 2 (for example, a micromotor 2a, a turbine 2b, an ablator 2c and a syringe 2s).

The dental unit 100 also comprises the water circuit 1 that supplies a liquid, which may come from a mains supply 3 or from other containers that are not illustrated, and a drain 10 for the liquid itself.

The water circuit 3 is divided into a plurality of branches 1s, 1a, 1b, 1c, each leading to one of the handpieces 2 present on the dental unit 100, necessitating a fluid supply and equipped with shutoff valves V.

The sterilizing/disinfecting method comprises the following steps, at least insofar as is relevant to the present invention:

introducing a disinfectant/sterilizing liquid in the water circuit 1 for a predetermined length of time;

draining the disinfectant/sterilizing liquid out of the circuit 1 through the circuit legs or branches 1a, 1b, 1c, 1s.

Obviously and as is well within the knowledge of experts in the trade familiar with methods of this kind, the shutoff valves V are opened and closed during these steps in order to allow the liquids to flow into the water circuit 1, be held there and then drained out.

In the method according to the invention, the step of introducing the disinfectant/sterilizing liquid is accompanied by a step of heating the liquid to a predetermined temperature higher than ambient temperature, using suitable means 4.

More specifically, the disinfectant/sterilizing liquid may be heated while the liquid is being introduced in the water circuit 1, that is to say, the step of introducing it and the step of heating it are simultaneous.

Alternatively, the step of heating the disinfectant/sterilizing liquid may be carried out just before the step of introducing the liquid, immediately upstream of the water circuit 1 (as described in more detail below).

If the disinfectant/sterilizing liquid consists of a first ingredient mixed with a second ingredient to form a third active compound, heating also has the purpose of enhancing the activity of the third active compound produced by the reaction between the two ingredients.

For example, the step of heating the disinfectant/sterilizing liquid may bring the liquid up to a temperature of between 30° and 550, and preferably between 35° and 45°.

Where the dental unit 100 comprises a microprocessor unit 5 that controls its main and auxiliary functions (which are not illustrated since they are of well-known type), the heating step, like all the other steps in the cycle, is controlled by the microprocessor unit 5 acting on the heating means 4. This enables the heating means 4 to be switched on and off in accordance with: the required temperature (which might vary from liquid to liquid); the temperature reached by the liquid (checked during inflow); the point in time the liquid starts flowing into the water circuit 1.

To further improve and speed up the sterilizing/disinfecting cycle, the step of introducing the disinfectant/sterilizing liquid might be preceded by a step of introducing a gaseous fluid designed to completely expel all the liquid previously inside the water circuit 1.

Preferably, but without restricting the scope of the invention, the step of introducing the heated disinfectant/sterilizing liquid in the water circuit 1 may comprise two or more separate sub-steps of introducing defined quantities of the liquid (to fill the circuit 1), alternated with steps of waiting for the next quantity of liquid to be introduced. Further, liquid inflow during each sub-step might be associated with a different temperature from the temperature associated with the sub-steps before and after it, so as to optimize the efficiency and speed of the sterilizing/disinfecting cycle: this might also depend on the type of product used. In brief, the purpose is to control the variables that influence the sterilizing/disinfecting action in order to optimize this action: the variables involved are hold time, operating temperature and concentration of the disinfectant/sterilizing product used.

The dental unit 100 that implements the method described above comprises the water circuit 1 for supplying liquids to each handpiece 2 and at least one first independent branch 6 connected to the water circuit 1 and supplying the water circuit 1 with at least one disinfectant/sterilizing liquid from a container 7.

The dental unit 100 comprises means 4 for heating the disinfectant/sterilizing liquid, the heating means 4 being mounted on the dental unit 100.

More specifically, the heating means 4 may be mounted between the container 7 and the first branch 6 that supplies the disinfectant/sterilizing liquid.

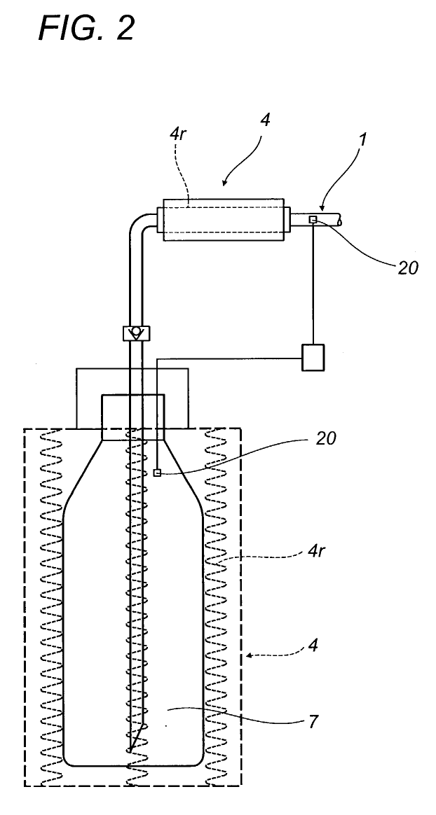

Looking in more detail, with reference to FIG. 2, the heating means 4 may be located on the first branch 6 that supplies the disinfectant/sterilizing liquid.

The presence on the dental unit 100, of the aforementioned microprocessor unit 5 that controls the main and auxiliary functions of the dental unit 100 itself means that the heating means 4 can be directly controlled by the microprocessor unit 5 (illustrated as a block) which switches the heating means on or off when the disinfectant/sterilizing liquid starts flowing into the water circuit 1.

Structurally, the heating means 4 may consist of one or more heating elements 4r wound around the first branch 6 that supplies the disinfectant/sterilizing liquid. Alternatively, the heating means 4 may consist of one or more heating elements 4r wound around the container 7 for the liquid in the dental unit 100.

The heating means 4 may also be equipped with sensor means 20 used to check the temperature of the disinfectant/sterilizing liquid and connected to the microprocessor unit 5 so as to enable the latter to control the heating means 4 according to the temperature of the liquid.

The method as described above achieves the aforementioned aims by improving and speeding up the sterilizing/disinfecting cycle thanks to the simple addition of a heating step which, by raising the temperature of the disinfectant/sterilizing liquid, enables the latter to be activated more quickly to sanitize dental unit conduits so that it needs to be held in the water circuit for shorter lengths of time compared to prior art methods.

The dental unit is not encumbered by additional equipment and thus the traditional structure of the dental unit remains practically unchanged.

The invention described can be subject to modifications and variations without thereby departing from the scope of the inventive concept. Moreover, all the details of the invention may be substituted by technically equivalent elements.

What is claimed is:

1. A method for sterilizing/disinfecting the water circuit (1) of a dental unit (100) at least comprising medical instruments, such as dental handpieces (2), and a water circuit (1) for supplying a fluid from a main supply (3) through corresponding circuit legs or branches (1a, 1b, 1c, 1s); the method comprising at least the following steps: introducing the disinfectant/sterilizing liquid in the water circuit (1) for a predetermined length of time and draining the disinfectant/sterilizing liquid out of the circuit (1) through the circuit legs or branches (1a, 1b, 1c, 1s), wherein the step of introducing the disinfectant/sterilizing liquid is accompanied by a step of heating the liquid to a predetermined temperature higher than ambient temperature, using suitable means (4).

2. The method according to claim 1, wherein the heating step is carried out during the step of introducing the disinfectant/sterilizing liquid, that is to say, inside the water circuit (1).

3. The method according to claim 1, wherein the step of heating the disinfectant/sterilizing liquid is carried out just before the step of introducing it, that is to say, upstream of the water circuit (1).

4. The method according to claim 1, where the disinfectant/sterilizing liquid consists of a first ingredient mixed with a second ingredient, wherein the step of heating the disinfectant/sterilizing liquid is carried out after the first and second ingredients have been mixed.

5. The method according to claim 1, wherein the step of heating the disinfectant/sterilizing liquid brings the liquid to a temperature of between 300 and 55°.

6. The method according to claim 1, wherein the step of heating the disinfectant/sterilizing liquid brings the liquid to a temperature of between 350 and 45°.

7. The method according to claim 1, where the dental unit comprises a microprocessor unit (5) to control the main and auxiliary functions of the dental unit (100), wherein the heating step is controlled by the microprocessor unit (5) acting on the heating means (4) so that the heating means (4) are switched on and off in accordance with the temperature reached by the liquid and the point in time the liquid starts flowing into the water circuit (1).

8. The method according to claim 1, wherein the step of introducing the disinfectant/sterilizing liquid in the water circuit (1) is preceded by a step of introducing a gaseous fluid so that all the user liquid previously inside the water circuit (1) is expelled.

9. The method according to claim 8, wherein the step of introducing the gaseous fluid includes a step of heating the gaseous fluid to the same temperature as that of the disinfectant/sterilizing liquid.

10. The method according to claim 1, wherein the step of introducing the disinfectant/sterilizing liquid in the water circuit (1) comprises two or more sub-steps of introducing defined quantities of the liquid for defined lengths of time, alternated with steps of waiting for the next quantity of liquid to be introduced.

11. The method according to claim 10, wherein each sub-step of introducing the disinfectant/sterilizing liquid in the water circuit (1) comprises a heating step to keep the liquid at the same temperature as in the previous or the following sub-step.

12. The method according to claim 10, wherein each sub-step of introducing the disinfectant/sterilizing liquid in the water circuit (1) comprises a step of heating the liquid to a different temperature from that of the previous or the following sub-step.

13. A dental unit comprising at least one main water circuit (1) for supplying a liquid to each handpiece (2) or accessory item of dental equipment present on the dental unit (100), and at least one independent circuit branch (6) connected to the water circuit (1) and supplying the water circuit (1) with at least one disinfectant/sterilizing liquid from a container (7), wherein the dental unit further comprises means (4) for heating the disinfectant/sterilizing liquid and mounted on the dental unit (100).

14. The dental unit according to claim 13, wherein the heating means (4) are positioned between the container (7) and the first branch (6) that supplies the disinfectant/sterilizing liquid.

15. The dental unit according to claim 13, wherein the heating means (4) are positioned on the first branch (6) that supplies the disinfectant/sterilizing liquid.

16. The dental unit according to claim 13, including a microprocessor unit (5) to control the main and auxiliary functions of the dental unit (100), wherein the heating means (4) are controlled by the microprocessor unit (5) which switches the heating means (4) on or off when the disinfectant/sterilizing liquid starts flowing into the water circuit (1).

17. The dental unit according to claim 13, wherein the heating means (4) consist of at least one heating element (4r) wound around the first branch (6) that supplies the disinfectant/sterilizing liquid.

18. The dental unit according to claim 13, wherein the heating means (4) consist of at least one heating element (4r) wound around the container (7) for the disinfectant/sterilizing liquid in the dental unit (100).

19. The dental unit according to claim 16, wherein the heating means (4) are equipped with sensor means (20) connected to the microprocessor unit (5) and used to check the temperature of the disinfectant/sterilizing liquid.
